(19) United States
(12) Patent Application Publication
(10) Pub. No.: US 2003/0144234 A1
(43) Pub. Date: Jul. 31, 2003

(54) Methods for the treatment of chronic pain and compositions therefor

(76) Inventors: Francis Paul Buxton, Morristown, NJ (US); Pamposh Ganju, London (GB); Christopher Robert Snell, Runcton Holme (GB); Chuanzheng Song, Warren, NJ (US)

Correspondence Address: THOMAS HOXIE, NOVARTIS, PATENT AND TRADEMARK DEPARTMENT, ONE HEALTH PLAZA 430/2, EAST HANOVER, NJ, 07936-1080, US

(21) Appl. No.: 10/231,425

(22) Filed: Aug. 28, 2002

Related U.S. Application Data
(60) Provisional application No. 60/315,898, filed on Aug. 30, 2001.

(51) Int. Cl. A61K 48/00; A61K 31/537; A61K 31/519

(52) U.S. Cl. 514/44; 514/265.1; 514/237.5

(57) ABSTRACT

The invention discloses cathepsin S as a suitable target for the development of new therapeutics to treat or ameliorate chronic pain. The invention relates to methods to treat and/or ameliorate chronic pain and pharmaceutical compositions therefor comprising modulators with inhibitory effect on cathepsin S enzyme activity and/or cathepsin S gene expression. The invention also relates to a method to identify compounds with therapeutic usefulness to treat chronic pain, comprising identifying compounds that can inhibit cathepsin S activity and/or gene expression which can also reverse the pathological effects of chronic pain in vivo.

### BACKGROUND OF THE INVENTION

Pain is a term that encompasses a spectrum of clinical states. Under normal conditions acute pain is beneficial and serves as a physiological warning for a potentially tissue-damaging situation. More persistent pain, usually associated with inflammation, can also be regarded as a normal protective response to mild tissue injury and resolves when the injury has healed. However, chronic pain occurs when the stimulus and pain are unrelated and the pain is no longer a protective mechanism. These types of pain syndromes (e.g. rheumatoid arthritis, cancer pain, neuropathic pain) are notoriously difficult to treat. It is estimated that 10-20% of the adult population suffers from chronic pain. To date, the main analgesics employed are based on opiates and non-steroidal anti-inflammatory drugs (NSAIDS) such as aspirin. Both classes of drugs can produce severe side-effects; NSAIDS can cause gastric ulceration and renal damage while opiates can cause nausea, constipation, confusion and dependency problems. Despite these disadvantages, no new class of analgesics have been discovered or developed recently; there is clearly a need for additional therapies for chronic pain.

Chronic pain states are characterised by a number of clinical features. As well as spontaneous pain, patients may exhibit hyperalgesia (a greatly exaggerated response to a noxious mechanical, hot, or cold stimulus), and allodynia (previously non-noxious stimuli are now perceived as painful). All these features result from a complex series of events involving changes in the function of sensory nerves in the periphery and in the processing of sensory information in the spinal cord and brain. These changes occur in response to direct neuronal damage or in response to mediators released during tissue damage or inflammation.

Broadly speaking, chronic pain syndromes can be defined as inflammatory (also known as nociceptive) or neuropathic. Chronic inflammatory pain, as its name suggests, occurs during conditions in which there is underlying inflammation such as rheumatoid arthritis, burns, muscle damage or surgical wounds. Knowledge of the mechanisms underlying inflammatory pain has advanced considerably over recent years and it is known to involve a variety of mediators and their activation and sensitization of the peripheral terminals of sensory nerves and the consequent longer term changes in reactivity of spinal cord neurons.

Chronic neuropathic pain is caused where there is a primary lesion or dysfunction of the nervous system and occurs, for example, during conditions such as trigeminal neuralgia, diabetic neuropathy, post-herpetic neuralgia, amputation or physical nerve damage. Chronic neuropathic pain results from damage to nerves by trauma, by diseases such as diabetes, herpes zoster, or late-stage cancer (see below), or by chemical injury (e.g. some anti-HIV drugs). It may also develop after amputation (including mastectomy), and is involved in some low-back pain. The mechanisms of chronic neuropathic pain are poorly understood but are thought to involve spontaneous firing of sensory nerves due to the novel expression of certain classes of ion channel, sprouting of sensory fibres into different layers of the spinal cord, and changes in the expression of various neurotransmitters and receptors in the sensory nerves and spinal cord. Traditionally chronic neuropathic pain has proven to be intractable and is resistant to the standard non-steroidal and opiate analgesics. There is therefore clearly an unmet clinical need for new analgesics to treat this type of pain.

Cancer pain is the most common chronic pain syndrome (with probably inflammatory and neuropathic components). It is estimated that one third of patients with advanced cancer will develop skeletal metastases, particularly in breast, prostate and lung cancer. Metastatic bone disease commonly results in bone pain that is usually located to a discrete area and is described as a deep, boring sensation that aches and burns, accompanied by episodes of stabbing discomfort. The mechanisms responsible for bone cancer pain are unknown but it probably involves structural damage, periosteal irritation and nerve entrapment. There is evidence for the disruption of normal bone metabolism and the production of inflammatory prostaglandins and cytokines. Current treatment of bone cancer pain rests with opiates but the doses required results in unacceptable side-effects and at least 20% of patients still have uncontrolled pain. Novel, well tolerated and effective analgesics are desired to optimise the quality of life of these patients (Coleman RE (1997) Cancer 80; 1588-1594).

Osteoarthritis pain is the most common form of chronic neuropathic pain (with probably inflammatory and neuropathic components) for which people visit general practitioners. Osteoarthritis is a chronic disease involving progressive structural changes in joint tissues, principally cartilage, synovium and subchondral bone. Typically, arthritic joints exhibit cartilage oedema and erosion, subchondral bone and synovial thickening, and formation of bony oesteophytes, all contributing to a deformation of the articular surface. The principal clinical symptom of osteoarthritis is pain, although the mechanisms underlying the chronic neuropathic pain in this condition are not understood.

Traditionally, attempts have been made to alleviate chronic neuropathic pain by directing therapeutic compounds to sensory fibers involved in pain signaling, e.g., the “C fiber”, (Woolf C. J. et al. (1995) J. Comp. Neurol. 360, 121-124.) or to the sensory fibers that transmit noxious information along the spinal cord (Dickenson A H. & Sullivan A. (1987) Neuropharmacol. 26; 1235-1238.). It has also been postulated that compounds may alleviate this pain by blocking mediator release (e.g. cytokines and bradykinins) from tissue during inflammation and/or blocking the receptors for these mediators (Dray A. & Urban L. (1996) Annu. Rev. Pharmacol. Toxicol. 36; 253-280.).

We have now surprisingly discovered that mRNA for cathepsin S, a lysosomal cysteine protease, is up regulated in animal models of chronic pain and that administration of cathepsin S inhibitors causes a reversal of mechanical hyperalgesia in these animals. Thus, cathepsin S can be used as a novel drug target for chronic pain. The invention also provides a method for identifying modulators that inhibit cathepsin S activity and/or inhibit cathepsin S gene expression and the use of such modulators for the treatment of chronic pain in human and veterinary patients. The invention also provides pharmaceutical compositions comprising said modulators.

### SUMMARY OF THE INVENTION

The instant application relates to the discovery that cathepsin S is a suitable target for the development of new therapeutics to treat or ameliorate chronic pain. Thus, in one aspect the invention relates to a method to identify modulators useful to treat or ameliorate chronic pain, including chronic neuropathic pain, comprising: a) assaying for the ability of a candidate modulator to inhibit the activity of cathepsin S and/or inhibit cathepsin S gene expression in vitro or in vivo and which can further include b) assaying for the ability of an identified inhibitory modulator to reverse the pathological effects observed in animal models of chronic pain and/or in clinical studies with subjects with chronic pain.

In another aspect, the invention relates to a method to treat or ameliorate chronic pain, including chronic neuropathic pain, comprising administering to a subject in need thereof an effective amount of a cathepsin S modulator, wherein said modulator, e.g., inhibits the enzyme activity of cathepsin S and/or inhibits cathepsin S gene expression in said subject. In one embodiment, the modulator is a compound belonging to a class of compounds referred to as N-heteroaryl-carbonitrile cathepsin inhibitors. In another embodiment, the modulator is the chemical compound [7-(2,2-Dimethyl-propyl)-6-thiophen-2-ylmethyl-7.H.-pyrrolo[2,3-.d.]pyrimidine-2-carbonitrile], in free or pharmaceutically acceptable salt forms, a substance designated herein as compound A. In a further embodiment, the modulator is the chemical compound 3-[(4-morpholinylcarbonyl)-phenylalanylamido]-1-fluoro-5-phenyl-2-pentanone, a substance designated herein as compound B. In another embodiment the modulator comprises any one or more substances selected from the group consisting of antisense oligonucleotides, triple helix DNA, ribozymes, RNA aptamers and double stranded RNA wherein said substances are designed to inhibit cathepsin S gene expression. In a further embodiment, the modulator comprises antibodies to cathepsin S or fragments thereof, wherein said antibodies can e.g., inhibit cathepsin S enzyme activity.

In another aspect, the invention relates to a method to treat or ameliorate chronic pain, including chronic neuropathic pain, comprising administering to a subject in need thereof a pharmaceutical composition comprising an effective amount of a cathepsin S modulator. In various embodiments, said pharmaceutical composition comprises any of the cathepsin S modulators discussed above.

In another aspect, the invention relates to a pharmaceutical composition comprising a cathepsin S modulator in an amount effective to treat or ameliorate chronic pain, including chronic neuropathic pain, in a subject in need thereof wherein said modulator, e.g., can inhibit the enzymatic activity of cathepsin S and/or inhibit cathepsin S gene expression. In one embodiment, said pharmaceutical composition comprises a compound belonging to a class of compounds referred to as N-heteroaryl-carbonitrile cathepsin inhibitors. In one embodiment, said pharmaceutical composition comprises the chemical compound designated herein as compound A, in free or pharmaceutically acceptable salt forms. In a further embodiment, said pharmaceutical composition comprises a substance designated herein as compound B. In another embodiment, said pharmaceutical composition comprises any one or more substances selected from the group consisting of antisense oligonucleotides, triple helix DNA, ribozymes, RNA aptamers or double stranded RNA directed to a nucleic acid sequence of cathepsin S wherein said substances are designed to inhibit cathepsin S gene expression. In a further embodiment, said pharmaceutical composition comprises antibodies to cathepsin S or fragments thereof, wherein said antibodies can, e.g., inhibit cathepsin S enzyme activity.

In another aspect, the invention relates to a method to diagnose subjects suffering from chronic pain who may be suitable candidates for treatment with cathepsin S modulators comprising detecting levels of this protein in a biological sample from said subject wherein subjects with increased levels compared to controls would be suitable candidates for cathepsin S modulator treatment.

In yet another aspect, the invention relates to a method to diagnose subjects suffering from chronic pain who may be suitable candidates for treatment with cathepsin S modulators comprising assaying mRNA levels of this protein in a biological sample from said subject wherein subjects with increased levels compared to controls would be suitable candidates for cathepsin S modulator treatment.

In yet another aspect, there is provided a method to treat or ameliorate chronic pain, including chronic neuropathic pain, comprising: (a) assaying for cathepsin S mRNA and/or protein levels in a subject; and (b) administering to a subject with increased levels of cathepsin S mRNA and/or protein levels compared to controls a cathepsin S modulator in an amount sufficient to treat or ameliorate the pathological effects of chronic pain.

In yet another aspect of the present invention there are provided assay methods and kits comprising the components necessary to detect expression of polynucleotides encoding cathepsin S or related regulatory polypeptides, or levels of cathepsin S or related regulatory polypeptides, or fragments thereof, in body tissue samples derived from a patient, such kits comprising, e.g., antibodies that bind to said polypeptides, or to fragments thereof, or oligonucleotide probes that hybridize with said polynucleotides. In a preferred embodiment, such kits also comprise instructions detailing the procedures by which the kit components are to be used.

The present invention also pertains to the use of a cathepsin S modulator in the manufacture of a medicament for the treatment or amelioration of chronic pain, including chronic neuropathic pain. In one embodiment, said cathepsin S modulator is compound A in free or pharmaceutically acceptable salt forms. In another embodiment, said cathepsin S modulator is compound B. In a further embodiment, said cathepsin S modulator comprises any one or more substances selected from the group consisting of antisense oligonucleotides, triple helix DNA, ribozymes, RNA aptamer and double stranded RNA wherein said substances are designed to inhibit cathepsin S gene expression. In yet a further embodiment, said cathepsin S modulator comprises one or more antibodies to cathepsin S, or fragments thereof, wherein said antibodies or fragments thereof can, e.g., inhibit cathepsin S enzyme activity.

The invention also pertains to a cathepsin S modulator for use as a pharmaceutical. In one embodiment, said cathepsin S modulator is compound A in free or pharmaceutically acceptable salt forms. In another embodiment, said cathepsin S modulator is compound B. In a further embodiment, said cathepsin S modulator comprises any one or more substances selected from the group consisting of antisense oligonucleotides, triple helix DNA, ribozymes, RNA aptamer and double stranded RNA wherein said substances are designed to inhibit cathepsin S gene expression. In yet a further embodiment, said cathepsin S modulator comprises one or more antibodies to cathepsin S, or fragments thereof, wherein said antibodies or fragments thereof can, e.g., inhibit cathepsin S enzyme activity.

### DESCRIPTION OF THE FIGURES

N/A

### DETAILED DESCRIPTION OF THE INVENTION

It is contemplated that the invention described herein is not limited to the particular methodology, protocols, and reagents described as these may vary. It is also to be understood that the terminology used herein is for the purpose of describing particular embodiments only, and is not intended to limit the scope of the present invention in any way.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices and materials are now described. All publications mentioned herein are incorporated by reference for the purpose of describing and disclosing the materials and methodologies that are reported in the publication which might be used in connection with the invention.

In practicing the present invention, many conventional techniques in molecular biology are used. These techniques are well known and are explained in, for example, Current Protocols in Molecular Biology, Volumes I, II, and III, 1997 (F. M. Ausubel ed.); Sambrook et al., 1989, Molecular Cloning: A Laboratory Manual, Second Edition, Cold Spring Harbor Laboratory Press, Cold Spring Harbor, N.Y.; DNA Cloning: A Practical Approach, Volumes I and II, 1985 (D. N. Glover ed.); Oligonucleotide Synthesis, 1984 (M. L. Gait ed.); Nucleic Acid Hybridization, 1985, (Hames and Higgins); Transcription and Translation, 1984 (Hames and Higgins eds.); Animal Cell Culture, 1986 (R. I. Freshney ed.); Immobilized Cells and Enzymes, 1986 (IRL Press); Perbal, 1984, A Practical Guide to Molecular Cloning; the series, Methods in Enzymology (Academic Press, Inc.); Gene Transfer Vectors for Mammalian Cells, 1987 (J. H. Miller and M. P. Calos eds., Cold Spring Harbor Laboratory); and Methods in Enzymology Vol. 154 and Vol.155 (Wu and Grossman, and Wu, eds., respectively).

As used herein and in the appended claims, the singular forms “a”, “an”, and “the” include plural reference unless the context clearly dictates otherwise. Thus, for example, reference to the “antibody” is a reference to one or more antibodies and equivalents thereof known to those skilled in the art, and so forth.

“Pathological effects of chronic pain” include, but are not limited to, hyperalgesia and allodynia.

The ability of a substance to “modulate” cathepsin S (e.g., a cathepsin S modulator) includes, but is not limited to, the ability of a substance to inhibit the enzymatic activity of cathepsin S and/or inhibit cathepsin S gene expression. Such modulation could also involve effecting the ability of other proteins to interact with cathepsin S, for example related regulatory proteins or proteins that are modified by cathepsin S.

“Nucleic acid sequence”, as used herein, refers to an oligonucleotide, nucleotide or polynucleotide, and fragments or portions thereof, and to DNA or RNA of genomic or synthetic origin that may be single or double stranded, and represent the sense or antisense strand.

The term “antisense” as used herein, refers to nucleotide sequences which are complementary to a specific DNA or RNA sequence. The term “antisense strand” is used in reference to a nucleic acid strand that is complementary to the “sense’ strand. Antisense molecules may be produced by any method, including synthesis by ligating the gene(s) of interest in a reverse orientation to a viral promoter which permits the synthesis of a complementary strand. Once introduced into a cell, this transcribed strand combines natural sequences produced by the cell to form duplexes. These duplexes then block either the further transcription or translation. The designation “negative” is sometimes used in reference to the antisense strand, and “positive” is sometimes used in reference to the sense strand.

As contemplated herein, antisense oligonucleotides, triple helix DNA, RNA aptamers, ribozymes and double stranded RNA are “directed to a nucleic acid sequence of cathepsin S” such that the nucleotide sequence of cathepsin S chosen will produce gene-specific inhibition of cathepsin S gene expression. For example, knowledge of the cathepsin S nucleotide sequence may be used to design an antisense molecule which gives strongest hybridization to the mRNA. Similarly, ribozymes can be synthesized to recognize specific nucleotide sequences of cathepsin S and cleave it (Cech. J. Amer. Med Assn. 260:3030 (1988). Techniques for the design of such molecules for use in targeted inhibition of gene expression is well known to one of skill in the art.

The term “cathepsin S” refers to any and all forms of this polypeptide including, but not limited to, partial forms, isoforms, precursor forms, the full length polypeptide, fusion proteins containing the cathepsin S sequence or fragments of any of the above, from human or any other species. The sequence of cathepsin S may be found in Genbank, Accession Number NM 004079. Homologs of cathepsin S, which would be apparent to one of skill in the art, are meant to be included in this definition. It is also contemplated that the term refers to cathepsin S isolated from naturally occurring sources of any species such as genomic DNA libraries as well as genetically engineered host cells comprising expression systems, or produced by chemical synthesis using, for instance, automated peptide synthesizers or a combination of such methods. Means for isolating and preparing such polypeptides are well understood in the art.

The term “sample” as used herein, is used in its broadest sense. A biological sample from a subject may comprise blood, urine or other biological material with which cathepsin S activity or gene expression may be assayed. A biological sample may include dorsal root ganglia from which total RNA may be purified for gene expression profiling using conventional glass chip microarray technologies such as Affymetrix chips, RT-PCR or other conventional methods.

As used herein, the term “antibody” refers to intact molecules as well as fragments thereof, such as Fa, F(ab′)₂, and Fv, which are capable of binding the epitopic determinant. Antibodies that bind cathepsin S polypeptides can be prepared using intact polypeptides or fragments containing small peptides of interest as the immunizing antigen. The polypeptides or peptides used to immunize an animal can be derived from the translation of RNA or synthesized chemically, and can be conjugated to a carrier protein, if desired. Commonly used carriers that are chemically coupled to peptides include bovine serum albumin and thyroglobulin. The coupled peptide is then used to immunize an animal (e.g., a mouse, a rat or a rabbit).

The term “humanized antibody” as used herein, refers to antibody molecules in which amino acids have been replaced in the non-antigen binding regions in order to more closely resemble a human antibody, while still retaining the original binding ability.

A “therapeutically effective amount” is the amount of drug sufficient to treat and/or ameliorate the pathological effects of chronic pain, including but not limited to, hyperalgesia.

“Related regulatory proteins” and “related regulatory polypeptides” as used herein refer to polypeptides involved in the regulation of cathepsin S which may be identified by one of skill in the art using conventional methods such as described herein.

Pain as defined herein includes chronic pain. “Chronic pain” includes inflammatory (nociceptive) and neuropathic pain as described above.

“Subject” refers to any human or nonhuman organism.

The invention is based on the surprising discovery that cathepsin S messenger RNA is up regulated in rat models of chronic neuropathic pain. This observation led to the additional discovery that cathepsin S inhibitors reverse the mechanical hyperalgesia produced in rats subjected to laboratory models of chronic pain. Thus, cathepsin S is a useful drug target for the development of therapeutics for the treatment of chronic pain, a disease state not previously known to involve cathepsin S.

Thus, in one aspect the invention relates to a method to identify modulators useful to treat or ameliorate chronic pain, including chronic neuropathic pain, comprising: a) assaying for the ability of a candidate modulator to inhibit the activity of cathepsin S and/or inhibit cathepsin S gene expression in vitro or in vivo and which can further include b) assaying for the ability of an identified inhibitory modulator to reverse the pathological effects observed in animal models of chronic pain and/or in clinical studies with subjects with chronic pain.

Conventional screening assays (both in vitro and in vivo) may be used to identify modulators that inhibit cathepsin S enzyme activity and/or inhibit cathepsin S gene expression. Cathepsin S activity levels can be assayed in a subject using a biological sample from the subject using conventional enzyme activity assay methods. Cathepsin S gene expression (e.g. mRNA levels) may also be determined using methods familiar to one of skill in the art, including, for example, conventional Northern analysis or commercially available microarrays. Additionally, the effect of test compounds' inhibition of cathepsin S and/or related regulatory protein levels can be detected with an ELISA antibody-based assay or fluorescent labeling reaction assay. These techniques are readily available for high throughput screening and are familiar to one skilled in the art.

Data gathered from these studies would be used to identify those modulators with therapeutic usefulness for the treatment of chronic pain as inhibitory substances could then be further assayed in conventional live animal models of chronic pain as described herein and/or in clinical trials with humans with chronic pain according to conventional methods to assess the ability of said compounds to ameliorate the pathological effects of chronic pain in vivo.

Candidate modulators for analysis according to the methods disclosed herein include chemical compounds known to possess cathepsin inhibitory activity as well as compounds whose effects on this protein at any level have yet to be characterized. Compounds known to possess cathepsin inhibitory activity could be directly assayed in the animal pain models described herein or in clinical trials.

Any compound with cathepsin inhibitory activity, and not necessarily only those compounds that specifically inhibit only cathepsin S, may prove to be useful therapeutics. For example, mixed cathepsin inhibitors (e.g., compounds that can inhibit cathepsins K or L, as well as S) would be useful in the instant invention.

Known cathepsin inhibitors, including cathepsin S specific inhibitors, useful in the instant invention include, but are not limited to, dipeptide nitriles described in published patent application WO99/24460 to Novartis Corporation; α-amino fluoro ketones, such as described in U.S. Pat. No. 4,518,528; peptides with fluoride free leaving groups as described in U.S. Pat. No. 5,374,623; compounds with heterocyclic leaving groups as described in U.S. Pat. No. 5,486,623; and compounds containing alkyl sulfonyls such as described in U.S. Pat. No. 6,030, 946. Additional references disclosing cathepsin inhibitors include: WO 00/49007; WO 00/49008; WO 97/40066; WO 96/40737; WO 01/19816; WO 00/55125 and WO 00/51998. For a review of the therapeutic potential of advances in cysteine protease inhibitor design see Veber, Daniel F; Thompson, Scott K. Curr. Opin. Drug Discovery Dev. (2000), 3(4), 362-369.

One particularly useful class of compounds is the 6-aryl-7H-pyrrolo-(2,3-d)-pyrimidine-2-carbonitrile cathepsin inhibitors which can be more generally referred to as N-heteroaryl-carbonitrile cathepsin inhibitors. One particularly useful compound of this class is a compound of Formula I, [7-(2,2-Dimethyl-propyl)-6-thiophen-2-ylmethyl-7.H.-pyrrolo[2,3-.d.]pyrimidine-2-carbonitrile] and pharmaceutically acceptable salts thereof, referred to herein as compound A (see Example 3).

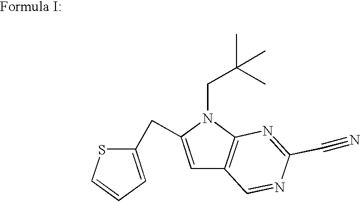

Compound A is further disclosed in PCT application serial number XXXX and may be synthesized as described therein. Briefly, 1-Prop-2-ynyl-2H-thiophene (15 mmol) is dissolved in DMF at room temperature under nitrogen atmosphere. To the solution, 5-bromo-4-(2,2-dimethyl-propylamino)-pyrimidine-2-carbonitrile (8 mmol), triethylamine (24 mmol), copper(I) iodide (0.8 mmol), and dichlorobis(triphenylphosphine)palladium(II) (0.4 mmol) are added successively. The mixture is heated at 80° C. under nitrogen atmosphere for 3 h. After cooling at room temperature, the mixture is diluted with H₂O and AcOEt and filtered with celite. The organic layer is taken, dried over MgSO₄ and evaporated in vacuo. The residue is purified by silica gel column chromatography (AcOEt: MeOH=20:1) to give 7-(2,2-dimethyl-propyl)-6-thiophen-2-ylmethyl-7H-pyrrolo[2,3-d]pyrimidine-2-carbonitrile in 64% yield.

Rf=0.50 (n-hexane:AcOEt=5:1); NMR: (CDCl₃):1.04 (s, 9H) 4.10 (s, 2H), 4.43 (s, 2H), 6.44 (s, 1H), 6.85-6.86 (m, 1H), 6.97-6.99 (m, 1H), 7.23-7.25 (m, 1H), 8.87 (s, 1H).

In addition, another particularly useful compound is a compound of Formula II, 3-[(4-morpholinylcarbonyl)-phenylalanylamido]-1-fluoro-5-phenyl-2-pentanone (disclosed in J. Clin. Invest 1993 Mar 91(3):1052-6 and U.S. Pat. No. 4,518,528).

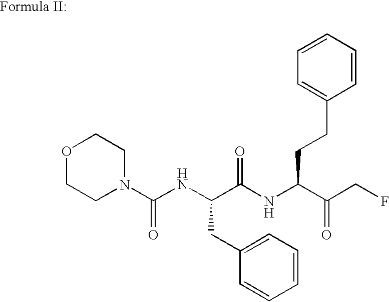

This compound is a mixed cathepsin inhibitor and is referred to herein as compound B (see Example 2) which can be prepared according to the scheme below.

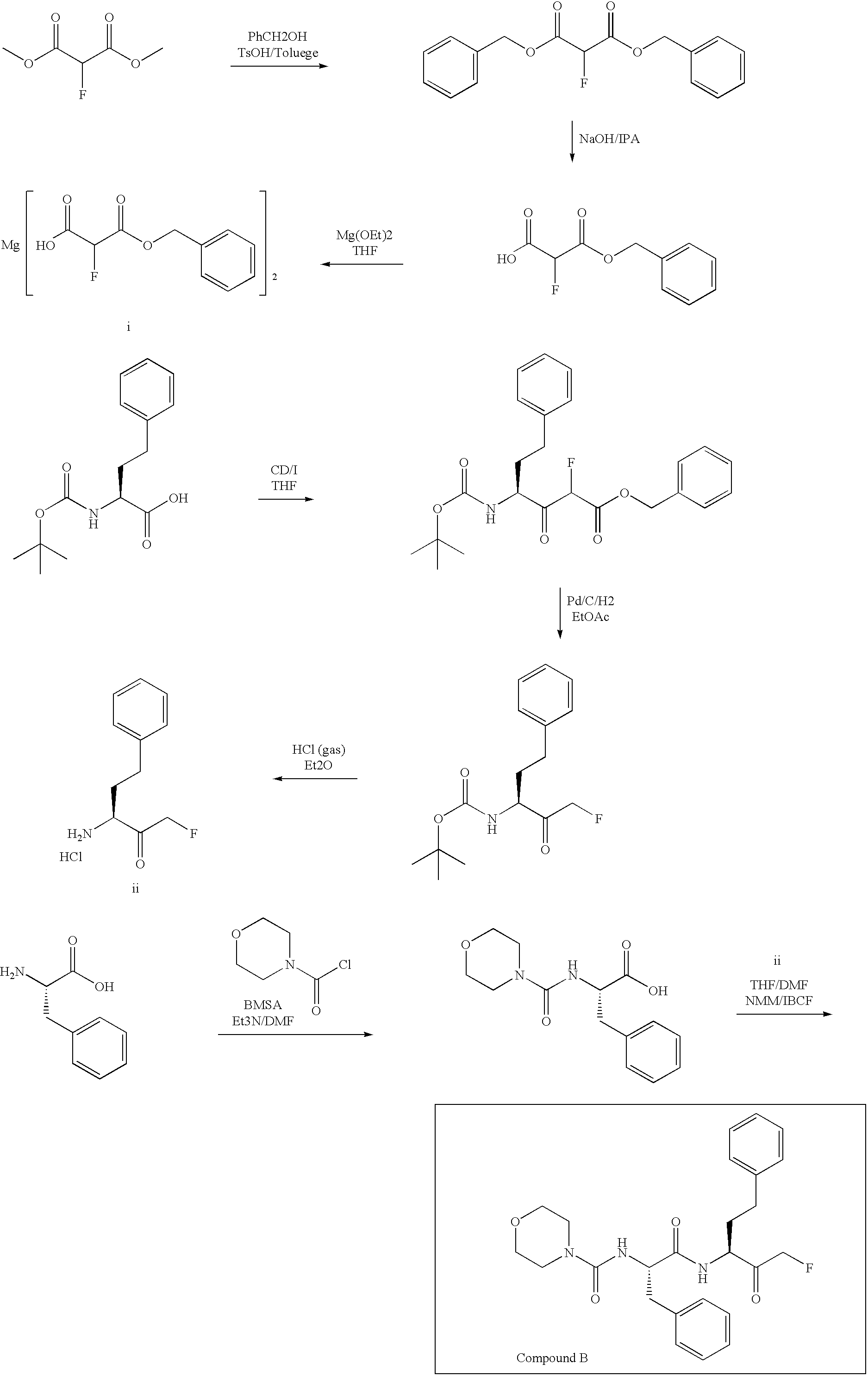

In another aspect, the invention relates to a method to treat or ameliorate chronic pain comprising administering to a subject in need thereof a pharmaceutical composition comprising an effective amount of a cathepsin S modulator. Such modulators include antibodies directed to the cathepsin S polypeptide or fragments thereof. In certain particularly preferred embodiments, the pharmaceutical composition comprises antibodies that are highly selective for human cathepsin S polypeptides or portions of human cathepsin S polypeptides. Antibodies to cathepsin S may cause the aggregation of the protein in a subject and thus inhibit or reduce the activity of the enzyme. Such antibodies may also inhibit or decrease cathepsin S activity, for example, by interacting directly with active sites or by blocking access of substrates to active sites. Cathepsin S antibodies may also be used to inhibit cathepsin S activity by preventing protein-protein interactions that may be involved in the regulation of cathepsin S and necessary for enzyme activity. Antibodies with inhibitory activity such as described herein can be produced and identified according to standard assays familiar to one of skill in the art.

Cathepsin S antibodies may also be used diagnostically. For example, one could use these antibodies according to conventional methods to quantitate levels of cathepsin S in a subject; increased levels would indicate chronic pain and the degree of severity of this condition. Thus, different cathepsin S levels would be indicative of various clinical forms or severity of chronic pain. Such information would also be useful to identify subsets of patients experiencing pain that may or may not respond to treatment with cathepsin S inhibitors. Similarly, it is contemplated herein that quantitating the message level of cathepsin S in a subject would be useful for diagnosis and determining appropriate pain therapy; subjects with increased mRNA levels of this protein compared to appropriate control individuals would be considered suitable candidates for treatment with cathepsin S inhibitors.

Thus in another aspect, the present invention relates to a diagnostic kit which comprises:

(a) a polynucleotide of cathepsin S or a fragment thereof;

(b) a nucleotide sequence complementary to that of (a);

(c) a cathepsin S polypeptide, or a fragment thereof; or

(d) an antibody to a cathepsin S polypeptide.

It will be appreciated that in any such kit, (a), (b), (c) or (d) may comprise a substantial component. It is also contemplated that said kit could comprise components (a)-(d) designed to detect levels of cathepsin S related regulatory proteins or proteins modified by cathepsin S as discussed herein.

Similarly, it is contemplated herein that monitoring cathepsin S protein levels or enzyme activity and/or detecting cathepsin S gene expression (mRNA levels) may be used as part of a clinical testing procedure, for example, to determine the efficacy of a given pain treatment regimen. For example, patients to whom pain medicine has been administered would be evaluated and the clinician would be able to identify those patients in whom cathepsin S levels, activity and/or gene expression levels are higher than desired (i.e. levels greater than levels in control patients not experiencing pain or in patients in whom pain has been sufficiently alleviated by clinical intervention). Based on these data, the clinician could then adjust the dosage, administration regimen or type of pain medicine prescribed. While the clinician can get an idea of the effectiveness of a particular pain medication by asking the patient how much pain he or she is experiencing, it is contemplated herein that monitoring patient levels of cathepsin S as described above would provide a quantitative assessment of a patient's pain level. In addition, monitoring the level of cathepsin S in a subject in such a way could be used to assess the level of pain experienced by nonresponsive patients (e.g. infants, comatose, burn patients). Such data could then be used by the clinician for determining the appropriate dosage, administration regimen or type of pain medication for such patients.

Factors for consideration for optimizing a therapy for a patient include the particular condition being treated, the particular mammal being treated, the clinical condition of the individual patient, the site of delivery of the active compound, the particular type of the active compound, the method of administration, the scheduling of administration, and other factors known to medical practitioners. The therapeutically effective amount of an active compound to be administered will be governed by such considerations, and is the minimum amount necessary for the treatment of chronic pain, preferably, chronic neuropathic pain.

Suitable antibodies to cathepsin S or related regulatory proteins can be obtained from a commercial source or produced according to conventional methods. For example, described herein are methods for the production of antibodies capable of specifically recognizing one or more differentially expressed gene epitopes. Such antibodies may include, but are not limited to polyclonal antibodies, monoclonal antibodies (mAbs), humanized or chimeric antibodies, single chain antibodies, Fab fragments, F(ab′)₂ fragments, fragments produced by a Fab expression library, anti-idiotypic (anti-Id) antibodies, and epitope-binding fragments of any of the above For the production of antibodies to the cathepsin S polypeptides discussed herein, various host animals may be immunized by injection with the polypeptides, or a portion thereof. Such host animals may include, but are not limited to, rabbits, mice, and rats. Various adjuvants may be used to increase the immunological response, depending on the host species, including, but not limited to, Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjuvants such as BCG (bacille Calmette-Guerin) and Corynebacterium parvum.

Polyclonal antibodies are heterogeneous populations of antibody molecules derived from the sera of animals immunized with an antigen, such as target gene product, or an antigenic functional derivative thereof. For the production of polyclonal antibodies, host animals such as those described above, may be immunized by injection with the polypeptides, or a portion thereof, supplemented with adjuvants as also described above.

Monoclonal antibodies, which are homogeneous populations of antibodies to a particular antigen, may be obtained by any technique which provides for the production of antibody molecules by continuous cell lines in culture. These include, but are not limited to the hybridoma technique of Kohler and Milstein, (1975, Nature 256:495-497; and U.S. Pat. No. 4,376,110), the human B-cell hybridoma technique (Kosbor et al., 1983, Immunology Today 4:72; Cole et al., 1983, Proc. Natl. Acad. Sci. USA 80:2026-2030), and the EBV-hybridoma technique (Cole et al., 1985, Monoclonal Antibodies And Cancer Therapy, Alan R. Liss, Inc., pp. 77-96). Such antibodies may be of any immunoglobulin class including IgG, IgM, IgE, IgA, IgD and any subclass thereof. The hybridoma producing the mAb of this invention may be cultivated in vitro or in vivo. Production of high titers of mAbs in vivo makes this the presently preferred method of production.

In addition, techniques developed for the production of “chimeric antibodies” (Morrison et al., 1984, Proc. Natl. Acad. Sci., 81:6851-6855; Neuberger et al., 1984, Nature, 312:604-608; Takeda et al., 1985, Nature, 314:452-454) by splicing the genes from a mouse antibody molecule of appropriate antigen specificity together with genes from a human antibody molecule of appropriate biological activity can be used. A chimeric antibody is a molecule in which different portions are derived from different animal species, such as those having a variable or hypervariable region derived from a murine mAb and a human immunoglobulin constant region.

Alternatively, techniques described for the production of single chain antibodies (U.S. Pat. No. 4,946,778; Bird, 1988, Science 242:423-426; Huston et al., 1988, Proc. Natl. Acad. Sci. USA 85:5879-5883; and Ward et al., 1989, Nature 334:544-546) can be adapted to produce differentially expressed gene-single chain antibodies. Single chain antibodies are formed by linking the heavy and light chain fragments of the Fv region via an amino acid bridge, resulting in a single chain polypeptide.

Most preferably, techniques useful for the production of “humanized antibodies” can be adapted to produce antibodies to the polypeptides, fragments, derivatives, and functional equivalents disclosed herein. Such techniques are disclosed in U.S. Pat. Nos. 5,932, 448; 5,693,762; 5,693,761; 5,585,089; 5,530,101; 5,910,771; 5,569,825; 5,625,126; 5,633,425; 5,789,650; 5,545,580; 5,661,016; and 5,770,429, the disclosures of all of which are incorporated by reference herein in their entirety.

Antibody fragments that recognize specific epitopes may be generated by known techniques. For example, such fragments include but are not limited to: the F(ab′)₂ fragments which can be produced by pepsin digestion of the antibody molecule and the Fab fragments which can be generated by reducing the disulfide bridges of the F(ab′)₂ fragments. Alternatively, Fab expression libraries may be constructed (Huse et al., 1989, Science, 246:1275-1281) to allow rapid and easy identification of monoclonal Fab fragments with the desired specificity.

Detection of the antibodies described herein may be achieved using standard ELISA, FACS analysis, and standard imaging techniques used in vitro or in vivo. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, (3-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I ³⁵S or ³H.

Particularly preferred, for ease of detection, is the sandwich assay, of which a number of variations exist, all of which are intended to be encompassed by the present invention. For example, in a typical forward assay, unlabeled antibody is immobilized on a solid substrate and the sample to be tested brought into contact with the bound molecule. After a suitable period of incubation, for a period of time sufficient to allow formation of an antibody-antigen binary complex, a second antibody, labeled with a reporter molecule capable of inducing a detectable signal, is added and incubated, allowing time sufficient for the formation of a ternary complex of antibody-antigen-labeled antibody. Any unreacted material is then washed away, and the presence of the antigen is determined by observation of a signal, or may be quantitated by comparing with a control sample containing known amounts of antigen. Variations on the forward assay include the simultaneous assay, in which both sample and antibody are added simultaneously to the bound antibody, or a reverse assay in which the labeled antibody and sample to be tested are first combined, incubated and added to the unlabeled surface bound antibody. These techniques are well known to those skilled in the art, and the possibility of minor variations will be readily apparent. As used herein, “sandwich assay” is intended to encompass all variations on the basic two-site technique. For the immunoassays of the present invention, the only limiting factor is that the labeled antibody be an antibody which is specific for the cathepsin S polypeptide or related regulatory protein, or fragments thereof.

The most commonly used reporter molecules are either enzymes, fluorophore- or radionuclide-containing molecules. In the case of an enzyme immunoassay an enzyme is conjugated to the second antibody, usually by means of glutaraldehyde or periodate. As will be readily recognized, however, a wide variety of different ligation techniques exist, which are well-known to the skilled artisan. Commonly used enzymes include horseradish peroxidase, glucose oxidase, beta-galactosidase and alkaline phosphatase, among others. The substrates to be used with the specific enzymes are generally chosen for the production, upon hydrolysis by the corresponding enzyme, of a detectable color change. For example, p-nitrophenyl phosphate is suitable for use with alkaline phosphatase conjugates; for peroxidase conjugates, 1,2-phenylenediamine or toluidine are commonly used. It is also possible to employ fluorogenic substrates, which yield a fluorescent product rather than the chromogenic substrates noted above. A solution containing the appropriate substrate is then added to the tertiary complex. The substrate reacts with the enzyme linked to the second antibody, giving a qualitative visual signal, which may be further quantitated, usually spectrophotometrically, to give an evaluation of the amount of polypeptide or polypeptide fragment of interest which is present in the serum sample.

Alternately, fluorescent compounds, such as fluorescein and rhodamine, may be chemically coupled to antibodies without altering their binding capacity. When activated by illumination with light of a particular wavelength, the fluorochrome-labeled antibody absorbs the light energy, inducing a state of excitability in the molecule, followed by emission of the light at a characteristic longer wavelength. The emission appears as a characteristic color visually detectable with a light microscope. Immunofluorescence and EIA techniques are both very well established in the art and are particularly preferred for the present method. However, other reporter molecules, such as radioisotopes, chemiluminescent or bioluminescent molecules may also be employed. It will be readily apparent to the skilled artisan how to vary the procedure to suit the required use.

The pharmaceutical compositions of the present invention may also comprise substances that inhibit the expression of cathepsin S at the nucleic acid level. Such molecules include ribozymes, antisense oligonucleotides, triple helix DNA, RNA aptamers and/or double stranded RNA directed to an appropriate nucleotide sequence of cathepsin S nucleic acid. These inhibitory molecules may be created using conventional techniques by one of skill in the art without undue burden or experimentation. For example, modifications (e.g. inhibition) of gene expression can be obtained by designing antisense molecules, DNA or RNA, to the control regions of the genes encoding the polypeptides discussed herein, i.e. to promoters, enhancers, and introns. For example, oligonucleotides derived from the transcription initiation site, e.g., between positions −10 and +10 from the start site may be used. Notwithstanding, all regions of the gene may be used to design an antisense molecule in order to create those which gives strongest hybridization to the mRNA and such suitable antisense oligonucleotides may be produced and identified by standard assay procedures familiar to one of skill in the art.

Similarly, inhibition of the expression of gene expression may be achieved using “triple helix” base-pairing methodology. Triple helix pairing is useful because it causes inhibition of the ability of the double helix to open sufficiently for the binding of polymerases, transcription factors, or regulatory molecules. Recent therapeutic advances using triplex DNA have been described in the literature (Gee, J. E. et al. (1994) In: Huber, B. E. and B. I. Carr, Molecular and Immunologic Approaches, Futura Publishing Co., Mt. Kisco, N.Y.). These molecules may also be designed to block translation of mRNA by preventing the transcript from binding to ribosomes.

Ribozymes, enzymatic RNA molecules, may also be used to inhibit gene expression by catalyzing the specific cleavage of RNA. The mechanism of ribozyme action involves sequence-specific hybridization of the ribozyme molecule to complementary target RNA, followed by endonucleolytic cleavage. Examples which may be used include engineered “hammerhead” or “hairpin” motif ribozyme molecules that can be designed to specifically and efficiently catalyze endonucleolytic cleavage of gene sequences, for example, the gene for cathepsin S.

Specific ribozyme cleavage sites within any potential RNA target are initially identified by scanning the target molecule for ribozyme cleavage sites which include the following sequences: GUA, GUU and GUC. Once identified, short RNA sequences of between 15 and 20 ribonucleotides corresponding to the region of the target gene containing the cleavage site may be evaluated for secondary structural features which may render the oligonucleotide inoperable. The suitability of candidate targets may also be evaluated by testing accessibility to hybridization with complementary oligonucleotides using ribonuclease protection assays.

Ribozyme methods include exposing a cell to ribozymes or inducing expression in a cell of such small RNA ribozyme molecules (Grassi and Marini, 1996, Annals of Medicine 28: 499-510; Gibson, 1996, Cancer and Metastasis Reviews 15: 287-299). Intracellular expression of hammerhead and hairpin ribozymes targeted to mRNA corresponding to at least one of the genes discussed herein can be utilized to inhibit protein encoded by the gene.

Ribozymes can either be delivered directly to cells, in the form of RNA oligonucleotides incorporating ribozyme sequences, or introduced into the cell as an expression vector encoding the desired ribozymal RNA. Ribozymes can be routinely expressed in vivo in sufficient number to be catalytically effective in cleaving mRNA, and thereby modifying mRNA abundance in a cell (Cotten et al., 1989 EMBO J. 8:3861-3866). In particular, a ribozyme coding DNA sequence, designed according to conventional, well known rules and synthesized, for example, by standard phosphoramidite chemistry, can be ligated into a restriction enzyme site in the anticodon stem and loop of a gene encoding a tRNA, which can then be transformed into and expressed in a cell of interest by methods routine in the art. Preferably, an inducible promoter (e.g., a glucocorticoid or a tetracycline response element) is also introduced into this construct so that ribozyme expression can be selectively controlled. For saturating use, a highly and constituently active promoter can be used. tDNA genes (i.e., genes encoding tRNAs) are useful in this application because of their small size, high rate of transcription, and ubiquitous expression in different kinds of tissues.

Therefore, ribozymes can be routinely designed to cleave virtually any mRNA sequence, and a cell can be routinely transformed with DNA coding for such ribozyme sequences such that a controllable and catalytically effective amount of the ribozyme is expressed. Accordingly the abundance of virtually any RNA species in a cell can be modified or perturbed.

Ribozyme sequences can be modified in essentially the same manner as described for antisense nucleotides, e.g., the ribozyme sequence can comprise a modified base moiety.

RNA aptamers can also be introduced into or expressed in a cell to modify RNA abundance or activity. RNA aptamers are specific RNA ligands for proteins, such as for Tat and Rev RNA (Good et al., 1997, Gene Therapy 4: 45-54) that can specifically inhibit their translation.

Gene specific inhibition of gene expression may also be achieved using conventional double stranded RNA technologies. A description of such technology may be found in WO 99/32619 which is hereby incorporated by reference in its entirety.

Antisense molecules, triple helix DNA, RNA aptamers and ribozymes of the present invention may be prepared by any method known in the art for the synthesis of nucleic acid molecules. These include techniques for chemically synthesizing oligonucleotides such as solid phase phosphoramidite chemical synthesis. Alternatively, RNA molecules may be generated by in vitro and in vivo transcription of DNA sequences encoding the genes of the polypeptides discussed herein. Such DNA sequences may be incorporated into a wide variety of vectors with suitable RNA polymerase promoters such as T7 or SP6. Alternatively, cDNA constructs that synthesize antisense RNA constitutively or inducibly can be introduced into cell lines, cells, or tissues.

Vectors may be introduced into cells or tissues by many available means, and may be used in vivo, in vitro or ex vivo. For ex vivo therapy, vectors may be introduced into stem cells taken from the patient and clonally propagated for autologous transplant back into that same patient. Delivery by transfection and by liposome injections may be achieved using methods that are well known in the art.

In addition to the above described methods for inhibiting the gene expression of cathepsin S, it is contemplated herein that one could identify and employ small molecules or other natural products to inhibit the transcription in vivo of the polypeptides discussed herein including, but not limited to, cathepsin S. For example, one of skill in the art could establish an assay for cathepsin S that can be easily applied to samples from the culture media of a cell line using conventional methods. Using this assay, cell lines would be screened to find ones that express cathepsin S. These cell lines would likely be of neuronal origin and would be cultured in, for example, 96 well plates. The closer the regulation of cathepsin S in the cell line to the expression in the dorsal root ganglia (DRG), the more likely it will be that small molecule modifiers of cathepsin S expression in the cell lines will also modify cathepsin S in DRG in vivo. A comparison of the effects of some known modifiers of gene expression e.g. dexamethasone, phorbol ester, heat shock on primary tissue DRG explants and the cell lines will allow the selection of the most appropriate cell line to use. The screen would then merely consist of culturing the cells for a set length of time with a different compound added to each well and then assaying for cathepsin S activity/mRNA level.

In order to facilitate the detection of cathepsin S in the assay described above, luciferase or other commercially available fluorescent protein could be genetically fused as an appropriate marker protein to the promoter of cathepsin S. Sequences upstream of the ATG of cathepsin S, i.e. the promoter of cathepsin S, can be identified from genomic sequence data by using the sequence from GenBank accession number NM_{—}004079 to BLAST against the NCBI genomic sequence. This gives at least 5 kb upstream of the ATG of cathepsin S that does not contain any unknown bases. Two pairs of nested PCR primers to amplify a fragment of 2 kb or longer from human genomic DNA can be readily designed and tested. The promoter fragment can be readily inserted into any promoter-less reporter gene vector designed for expression in human cells (e.g. Clontech promoter-less enhanced fluorescent protein vector pECFP-1, pEGFP-1, or PEYFP). The screen would then consist of culturing the cells for an appropriate length of time with a different compound added to each well and then assaying for reporter gene activity. Promising compounds would then be assayed for effects on cathepsin S activity and/or mRNA level in vivo using the in vivo models of chronic pain previously described. Additional method details such as appropriate culturing time, culture conditions, reporter assays and other methodologies that can be used to identify small molecules or other natural products useful to inhibit the transcription of cathepsin S in vivo would be familiar to one of skill in the art.

In addition, the cDNA and/or protein of cathepsin S can be used to identify other proteins, e.g. receptors, that are modified by cathepsin S in neurons from DRG or other tissues in the nervous system. Proteins thus identified can be used for drug screening to treat chronic pain. To identify these genes that are downstream of cathepsin S, it is contemplated, for example, that one could use conventional methods to treat animals in chronic pain models with a specific cathepsin S inhibitor, sacrifice the animals, remove DRG and isolate total RNA from these cells and employ standard microarray assay technologies to identify message levels that are altered relative to a control animal (animal to whom no drug has been administered).

Based on the knowledge that cathepsin S is upregulated in chronic pain states, conventional in vitro or in vivo assays may be used to identify possible genes that lead to over expression of cathepsin S. These related regulatory proteins encoded by genes thus identified can be used to screen drugs that might be potent therapeutics for the treatment of chronic pain. For example, a conventional reporter gene assay could be used in which the promoter region of cathepsin S is placed upstream of a reporter gene, the construct transfected into a suitable neuronal cell (for example, a neuroblastoma cell line) and using conventional techniques, the cells assayed for an upstream gene that causes activation of the cathepsin S promoter by detection of the expression of the reporter gene.

It is contemplated herein that one can inhibit the function and/or expression of a gene for a related regulatory protein or protein modified by cathepsin S as a way to treat chronic pain by designing, for example, antibodies to these proteins and/or designing inhibitory antisense oligonucleotides, triple helix DNA, ribozymes and RNA aptamers targeted to the genes for such proteins according to conventional methods. Pharmaceutical compositions comprising such inhibitory substances for the treatment of chronic pain are also contemplated.

The pharmaceutical compositions disclosed herein useful for treating and/or ameliorating chronic pain, including chronic neuropathic pain, are to be administered to a patient at therapeutically effective doses to treat or ameliorate symptoms of such disorders. A therapeutically effective dose refers to that amount of the compound sufficient to result in amelioration of pain symptoms of chronic pain based on, for example, use of the McGill pain score (Melzack, R. Pain (1975) September 1(3):277-299).

The inhibitory substances of the present invention can be administered as pharmaceutical compositions. Such pharmaceutical compositions for use in accordance with the present invention may be formulated in a conventional manner using one or more physiologically acceptable carriers or excipients.

Thus, the compounds and their physiologically acceptable salts and solvates may be formulated for administration by inhalation or insufflation (either through the mouth or the nose) or topical, oral, buccal, parenteral or rectal administration.

For oral administration, the pharmaceutical compositions may take the form of, for example, tablets or capsules prepared by conventional means with pharmaceutically acceptable excipients such as binding agents (e.g., pregelatinized maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

Preparations for oral administration may be suitably formulated to give controlled release of the active compound.

For buccal administration the compositions may take the form of tablets or lozenges formulated in conventional manner.

For administration by inhalation, the compounds for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration.

Pharmaceutical compositions suitable for use in the invention include compositions wherein the active ingredients are contained in an effective amount to achieve the intended purpose. The determination of an effective dose is well within the capability of those skilled in the art.

For any compound, the therapeutically effective dose can be estimated initially either in cell culture assays, e.g., of neoplastic cells, or in animal models, usually mice, rabbits, dogs, or pigs. The animal model may also be used to determine the appropriate concentration range and route of administration. A dose may be formulated in animal models to achieve a circulating plasma concentration range that includes the IC₅₀ (i.e., the concentration of the test compound that achieves a half-maximal inhibition of symptoms). Such information can then be used to determine useful doses and routes for administration in humans.

A therapeutically effective dose refers to that amount of active ingredient, for example, compound A or compound B, antisense oligonucleotides, triple helix DNA, ribozymes, RNA aptamer and double stranded RNA designed to inhibit cathepsin S gene expression, antibodies to cathepsin S or related regulatory proteins or fragments thereof, useful to treat and/or ameliorate the pathological effects of chronic pain. Therapeutic efficacy and toxicity may be determined by standard pharmaceutical procedures in cell cultures or experimental animals, e.g., ED50 (the dose therapeutically effective in 50% of the population) and LD50 (the dose lethal to 50% of the population). The dose ratio between toxic and therapeutic effects is the therapeutic index, and it can be expressed as the ratio, LD50/ED50. Pharmaceutical compositions that exhibit large therapeutic indices are preferred. The data obtained from cell culture assays and animal studies is used in formulating a range of dosage for human use. The dosage contained in such compositions is preferably within a range of circulating concentrations that include the ED50 with little or no toxicity. The dosage varies within this range depending upon the dosage form employed, sensitivity of the patient, and the route of administration.

The exact dosage will be determined by the practitioner, in light of factors related to the subject that requires treatment. Dosage and administration are adjusted to provide sufficient levels of the active moiety or to maintain the desired effect. Factors that may be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week, or once every two weeks depending on half-life and clearance rate of the particular formulation.

Normal dosage amounts may vary from 0.1 to 100,000 micrograms, up to a total dose of about 1 g, depending upon the route of administration. Guidance as to particular dosages and methods of delivery is provided in the literature and generally available to practitioners in the art. Those skilled in the art will employ different formulations for nucleotides than for proteins or their inhibitors. Similarly, delivery of polynucleotides or polypeptides will be specific to particular cells, conditions, locations, etc. Pharmaceutical formulations suitable for oral administration of proteins are described, e.g., in U.S. Pat. Nos. 5,008,114; 5,505,962; 5,641,515; 5,681,811; 5,700,486; 5,766,633; 5,792,451; 5,853,748; 5,972,387; 5,976,569; and 6,051,561.

The following examples further illustrate the present invention and are not intended to limit the invention.

### EXAMPLE 1

### RNA Isolation and Expression Profiling of Cathepsin S in Animal Models of Chronic Pain

In vivo Animal Models of Chronic Pain Include the Following:

Chronic Inflammatory Pain Model:

The Complete Freund's Adjuvant-induced mechanical hyperalgesia may be used as a model of chronic inflammatory pain (Stein, C. et al. Pharmacol. Biochem. Behav. (1988) 31:445-451). In this model, typically a male Sprague-Dawley orWistar rat (200-250 g) receives an intraplantar injection of 25 μl complete Freund's adjuvant into one hind paw. A marked inflammation occurs in this hind paw. Drugs are generally administered for evaluation of efficacy, 24 hours after the inflammatory insult, when mechanical hyperalgesia is considered fully established.

Chronic Neuropathic Pain Models:

Two animal models of chronic neuropathic pain may be used that involve some form of peripheral nerve damage. In the Seltzer model (Seltzer et al. (1990) Pain 43: 205-218) rats are anaesthetised and a small incision made mid-way up one thigh (usually the left) to expose the sciatic nerve. The nerve is carefully cleared of surrounding connective tissues at a site near the trochanter just distal to the point at which the posterior biceps semitendinosus nerve branches off the common sciatic nerve. A 7-0 silk suture is inserted into the nerve with a ⅜ curved, reversed-cutting mini-needle, and tightly ligated so that the dorsal ⅓ to ½ of the nerve thickness is held within the ligature. The muscle and skin are closed with sutures and clips and the wound dusted with antibiotic powder. In sham animals the sciatic nerve is exposed but not ligated and the wound closed as in nonsham animals.

In the Chronic Constriction Injury (CCl) model (Bennett, G. J. and Xie, Y. K. Pain (1988) 33: 87-107) rats are anaesthetised and a small incision is made mid-way up one thigh (usually the left) to expose the sciatic nerve. The nerve is cleared of surrounding connective tissue and four ligatures of 4/0 chromic gut are tied loosely around the nerve with approximately 1 mm between each, so that the ligatures just barely constrict the surface of the nerve. The wound is closed with sutures and clips as described above. In sham animals the sciatic nerve is exposed but not ligated and the wound closed as in nonsham animals.

In contrast to the Seltzer and CCl models, the Chung model involves ligation of the spinal nerve. (Kim, S. O. and Chung, J. M. Pain (1992): 50:355-363). In this model, rats are anesthetized and placed into a prone position and an incision is made to the left of the spine at the L4-S2 level. A deep dissection through the paraspinal muscles and separation of the muscles from the spinal processes at the L4-S2 level will reveal part of the sciatic nerve as it branches to form the L4, L5 and L6 spinal nerves. The L6 transverse process is carefully removed with a small rongeur enabling visualization of these spinal nerves. The L5 spinal nerve is isolated and tightly ligated with 7-0 silk suture. The wound is closed with a single muscle suture (6-0 silk) and one or two skin closure clips and dusted with antibiotic powder. In sham animals the L5 nerve is exposed as before but not ligated and the wound closed as before.

Behavioral index

In all chronic pain models (inflammatory and neuropathic) mechanical hyperalgesia is assessed by measuring paw withdrawal thresholds of both hindpaws to an increasing pressure stimulus using an Analgesymeter (Ugo-Basile, Milan). Mechanical allodynia is assessed by measuring withdrawal thresholds to non-noxious mechanical stimuli applied with von Frey hairs to the plantar surface of both hindpaws. Thermal hyperalgesia is assessed by measuring withdrawal latencies to a noxious thermal stimulus applied to the underside of each hindpaw. With all models, mechanical hyperalgesia and allodynia and thermal hyperalgesia develop within 1-3 days following surgery and persist for at least 50 days. For the assays described herein, drugs may be applied before and after surgery to assess their effect on the development of hyperalgesia, particularly approximately 14 days following surgery, to determine their ability to reverse established hyperalgesia.

The percentage reversal of hyperalgesia is calculated as follows:

$$\% reversal = \frac{postdose threshold - predose threshold}{naive threshold - predose threshold} \times 100$$

In the experiments disclosed herein, Wistar rats (male) are employed in the pain models described above. Rats weigh approximately 120-140 grams at the time of surgery. All surgery is performed under enflurane/O₂ inhalation anaesthesia. In all cases the wound is closed after the procedure and the animal allowed to recover. In all pain models employed, after a few days in all but the sham operated animals, a marked mechanical and thermal hyperalgesia and allodynia develops in which there is a lowering of pain threshold and an enhanced reflex withdrawal response of the hind-paw to touch, pressure or thermal stimuli. After surgery the animals also exhibit characteristic changes to the affected paw. In the majority of animals the toes of the affected hind paw are held together and the foot turned slightly to one side; in some rats the toes are also curled under. The gait of the ligated rats varies, but limping is uncommon. Some rats are seen to raise the affected hind paw from the cage floor and to demonstrate an unusual rigid extension of the hind limb when held. The rats tend to be very sensitive to touch and may vocalise. Otherwise the general health an condition of the rats is good.

RNA Extraction from DRG Taken from Rats Subjected to Chronic Neuropathic Pain Models (Seizer, CCl and Chung):

L4 and L5 DRG ipsilateral to the nerve injury are dissected at days 14, 21 and 50 after surgery from rat models of neuropathic pain according to standard methods. Total RNA samples are then prepared from the dissected DRG tissues according to the acid guanidinium thiocyanate-phenol-chloroform extraction method (Chomczynski and Sacchi Anal. Biochem., (1987) 162:156-159; Chomczynski, P., Biotechniques, 15: 532-537 (1993)). The yield is approximately 1 μg total RNA per DRG. Reverse transcription (primer: T7prFB 5′-aaacgacggcacftcgaaattaatacgactcactatagggagacc.t₃₀-3′) and preparation of biotin labeled probes from the total RNA samples are carried out according to conventional methods (Lockhart D J et al. Nat Biotechnol. 14:1675-80 (1996); Mahadevappa M, Warrington J A. Nat Biotechnol. 17:1134-6 (1999)). 5 ug total RNA is the approximate yield from 15-35 μg labeled biotin RNA. Each labeled RNA is hybridized to two Affymetrix rat U34A GeneChips using standard methods. The images are then analyzed using the Affymetrix GeneChip software to obtain the normalized average difference value as the measurement of RNA expression level (Affymetrix, Santa Clara, Calif.). After exporting the data as text files the data is analyzed.

Table 1 summarizes the relative mRNA levels of cathepsin S as measured in arbitrary units (with standard deviation in parentheses) from the Affymetrix RNA profiling experiments (N.D.: Not determined). Data indicate that cathepsin S messenger RNA is upregulated relative to the sham equivalents in the CCl model at days 14 and 21 and in the Seltzer model at days 14, 21 and 50. These time points reflect conditions when neuropathic hyperalgesia is well established and long lasting.

TABLE 1
Cathepsin S mRNA is upregulated in animal
models of chronic neuropathic pain

| | Day 14 | Day 21 | Day 50 |
|---|---|---|---|
| Sham | 257 (64) | 218 (31) | 197 (28) |
| Seltzer Model | 450 (119) | 444 (75) | 419 (40) |
| CCI Model | 639 (24) | 616 (64) | N.D. |

Cathepsin S is Expressed in Human DRG:

The human cathepsin S sequence (Genbank Accession number NM 004079 was used to search a proprietary sequence database constructed by sequencing 60,000 clones from a normalized human DRG cDNA library (Gibco BRL, Rockville, Md., USA). Two clones, fga0000208999 and fga00000206288, have identical sequences to NM 004079. Therefore, one can conclude that cathepsin S is also expressed in human DRG. However, in order to confirm that cathepsin S is expressed in human DRG, real time PCR is performed according to the following procedure:

Based on the sequence of the human cathepsin S gene from Genbank, (accession number NM 004079), two pairs of primers for real time PCR are designed.

| Primer | | Position Within |
|---|---|---|
| Name | Sequence | Sequence |
| cathS1F | GCAATGGTGGCTTCATGACA | 425 |
| cathS1R | ACATTTCTGATCCATGGCTTTGT | 525 |
| cathS2F | TGGGAATGCACTCATACGATCT | 89 |
| cathS2R | CCACTGGCTGGGAACTCTCA | 189 |

Oligonucleotides are made by Sigma-Genosys (The Woodlands, Tex.). Total RNA from normal human DRG is obtained from GeneWiz Inc. (New York, N.Y.). Synthesis of cDNA from total RNA is performed using the TaqMan Reverse Transcription Reagents Kit (Part No. N₈O₈-0234) from PE Applied Biosystems (Foster City, Calif.) according to the manufacturers protocol. Each 100 μL reaction contains 2 μg of total RNA, 1×TaqManRT buffer, 5.5 mM MgCl₂, 500 μM each dNTP, 2.5 μM random hexamers, 0.4U/μL RNase Inhibitor, and 1.25U/μL MultiScribe Reverse Transcriptase. Reactions are incubated at 25° C. for 10 minutes, 48° C. for 45 minutes, and then 75° C. for 5 minutes.

Quantitative PCR is performed using the SYBR Green PCR Core Reagents Kit (Part No. 4304886) from PE Applied Biosystems (Foster City, Calif.) according to the manufacturers protocol. Incubations and detection are done using the ABI Prism 7700 Sequence Detection System from PE Applied Biosystems. Each 50 μL reaction contains 5 μL of template cDNA from the reverse transcription reaction described above, 1×SYBR Green PCR buffer, 3 mM MgCl₂, 200 μM dATP, 200 μM dCTP, 200 μM dGTP, 400 μM dUTP, 0.025U/μL AmpliTaq Gold, 0.01 U/μL AmpErase UNG, and 50 nM each forward and reverse primer. Reactions are incubated at 500C for 2 minutes, 95° C. for 10 minutes, followed by 40 cycles of 95° C. for 15 seconds and 60° C. for 1 minute.

A transcript is detectably amplified with both primer pairs at least 7.8 cycles before a transcript is detected in the “no reverse transcriptase” controls.

Cycle at which message
is just detectable

| | no reverse | plus reverse |
|---|---|---|
| Primers | transcriptase | transcriptase |
| cathS-1 F and R | 34.9 | 27.1 +/− 0.30% |
| cathS-2 F and R | 40 | 26.5 +/− 0.09% |

The products of the PCR amplification are analyzed on an acrylamide gel according to conventional methods. Both “plus reverse transcriptase” reactions produced a single band that is 101 bp long as expected. These data confirm that cathepsin S is detectably expressed in human DRG.

### EXAMPLE 2

### Therapeutic Effect of Cathepsin S Inhibitors in Animal Models of Chronic Pain: Compound B

Based on the data indicating that cathepsin S is upregulated in animal models of chronic neuropathic pain, the ability of 3-[(4-morpholinylcarbonyl)-phenylalanylamido]-1-fluoro-5-phenyl-2-pentanone (compound B), prepared as described above) which is known to inhibit cysteine and serine proteases, including cathepsins S, in vitro (U.S. Pat. No. 4,518,528), was studied for its ability to reverse the pathological effects produced in chronic pain models.

Rats are subjected to the surgical procedures according to the CCl chronic pain model described above. Paw withdrawal thresholds are measured prior to surgery, 14 days later when hyperalgesia is established and then at 1, 3 and 6 hours following a single oral gavage dose of compound B. Results are provided in Table 2, below. Each time point represents data from 6 animals per group. Vehicle control: PEG/methyl cellulose (0.5%)+Tween 80 (0.25%)(20:80), 1 ml p.o.

TABLE 2
The effect of compound B on established mechanical
hyperalgesia in the CCI model of chronic neuropathic pain:
single daily oral dose
% Reversal of Predose Hyperalgesia

| Time | | | | |
|---|---|---|---|---|
| point | | 10 mg/kg | 30 mg/kg | 100 mg/kg |
| (hrs) | Vehicle | Cmpd B | Cmpd B | Cmpd B |
| 1 | −2.08 | 26.16 | 40.97 | 76.49 |
| 3 | 0.00 | 38.19 | 38.89 | 55.06 |
| 6 | 1.85 | 18.29 | 13.89 | 29.76 |

Data disclosed herein indicate that compound B can reverse the mechanical hyperalgesia induced in the CCl model of neuropathic pain in a dose dependent manner with a D50 in the range 30-100 mg/kg, with up to 70% reversal at 100 mg/kg at 1 hour post dosing. Additional studies indicate that compound B is also acutely effective at reversing mechanical hyperalgesia in the CCl model at 30 mg/kg when given orally, twice daily throughout the development of the hyperalgesia model (Table 3). In the twice daily study, paw withdrawal thresholds were measured prior to surgery and then daily 3 hours following the second oral dosing with compound B. The last dose was given on day 13. Each time point in Table 3 represents data from 6 animals per group. Vehicle control: PEG/methyl cellulose (0.5%)+Tween 80 (0.25%)(20:80), 1 ml p.o.

Interestingly, data for day 15 indicate that hyperalgesia returns after curtailment of the treatment with this cysteine cathepsin inhibitor, suggesting that the inhibitor does not affect development of hyperalgesia.

TABLE 3
The effect of compound B on established mechanical hyperalgesia in the CCI
model of chronic neuropathic pain: twice daily oral dosing

| | Day post | | | | | | | | | |
|---|---|---|---|---|---|---|---|---|---|---|
| | surgery | 1 | 2 | 3 | 4 | 6 | 8 | 10 | 13 | 15 |
| Mean Paw pressure | Control | 99.17 | 59.17 | 59.17 | 58.30 | 60.00 | 59.17 | 55.83 | 58.33 | 59.17 |
| withdrawal threshold (g) | Compound B | 100.00 | 70.83 | 79.17 | 80.00 | 80.80 | 80.00 | 77.50 | 79.17 | 60.83 |

Studies also indicate that compound B also reverses mechanical hyperalgesia produced in rats in the FCA-induced model of chronic inflammatory pain 24 hours after induction, but to a lesser extent than that seen in the chronic neuropathic models (maximal reversal of 46% at 100 mg/kg p.o., data not shown). While cathepsin S mRNA levels were not assayed in chronic inflammatory pain models, the efficacy of cathepsin S inhibitors in the chronic inflammatory pain model indicates that cathepsin S inhibitors such as disclosed herein would be effective therapeutics for the treatment and/or amelioration of chronic inflammatory pain.

While it is possible that compound B is acting on one or more of cathepsins S, K or L, its ability to reduce hyperalgesia in chronic pain models is most likely due to its ability to inhibit cathepsin S activity since studies with cathepsin K and L inhibitors indicate that these particular types of cathepsin inhibitors are not effective at reversing the hyperalgesia produced in animal models of chronic pain (our unpublished data). Thus, one may use a pharmaceutical composition comprising compound B to treat chronic pain.

### EXAMPLE 3

### Therapeutic Effect of Cathepsin S Inhibitors in Animal Models of Chronic Pain: Compound A

The ability of another mixed cathepsin inhibitor, compound A or [7-(2,2-Dimethyl-propyl)-6-thiophen-2-ylmethyl-7.H.-pyrrolo[2,3-.d.]pyrimidine-2-carbonitrile] on reversing the pathological effects produced in chronic pain models was also studied. Data from the Seltzer model of chronic neuropathic pain indicate that this compound can reverse hyperalgesia with a D50 between 3-10 mg/kg (Table 4).

TABLE 4
The effect of compound A on established mechanical hyperalgesia
in the Seltzer model of chronic neuropathic pain: single oral dose

| Time | | | | | |
|---|---|---|---|---|---|
| point | | 3 mg/kg | 10 mg/kg | 30 mg/kg | 30 mg/kg |
| (hrs) | Vehicle | Cmpd A | Cmpd A | Cmpd A | Cmpd B |
| 1 | 0.00 | 1.32 | 8.63 | 31.15 | 33.47 |
| 3 | 0.30 | 3.64 | 52.94 | 68.32 | 41.57 |
| 6 | −2.08 | −1.06 | 17.63 | 23.05 | 11.97 |

As described above, rats in this experiment are subjected to the surgical procedures according to the CCl chronic pain model. Paw withdrawal thresholds are measured prior to surgery, 14 days later when hyperalgesia is established and then following a single oral dosing with compound A. Each point represents data from 6 animals per group. Vehicle control: PEG/methyl cellulose (0.5%)+Tween 80 (0.25%)(20:80), 1 ml p.o.

Compound A has mixed cathepsin S/K inhibitory activity. Since data indicate that a specific cathepsin K inhibitor as well as a compound with inhibitory effects on cathepsin K and L are both unable to reverse hyperalgesia in models of neuropathic pain (our unpublished data), the potent reversal of hyperalgesia by compound A is due to inhibition of cathepsin S, thus verifying this enzyme as a therapeutic target for chronic neuropathic pain as well as chronic inflammatory pain.

### EXAMPLE 4

### In vitro Assay of Enzyme Activity

As discussed above, in order to identify compounds with potential therapeutic usefulness against chronic pain, a candidate compound with unknown effects on cathepsin S activity should first be assayed to determine effect on the activity of this enzyme. There are many ways of screening for cathepsin S enzyme activity using conventional methods, for example using fluorescent and other substrates. The assay described below is a homogeneous plate assay using a quenched fluorescent peptide substrate. The cleavage of the peptide by the enzyme results in a fluorescent product. Inhibiting the production of the fluorescence indicates enzyme inactivity and thus compound efficacy.

The enzyme is recombinant human cathepsin S (Novartis Pharmaceuticals Corporation, Summit, N.J.) expressed in either baculovirus or yeast, purified and stored frozen in the latent form (3.8 μM) according to conventional methods.

On the day of the assay the enzyme is activated by dilution 1:600 in assay buffer (88 mM KH₂PO₄, 12 mM Na₂HPO₄, 1.33 mM EDTA, 2.7 mM DTT, 0.03% Brij, pH 5.8) and then incubated 30 minutes on ice. The activated enzyme is used directly at 1,600 pM. Z-val-val-arg-AMC (50 mg, Bachem, King of Prussia, PA, I-1540), the cathepsin S substrate, is dissolved in 12.6 ml DMSO to give a final concentration of 6 mM. This is then diluted with assay buffer to give a final concentration of 150 μM. There is a final 1:4 dilution of the substrate upon addition to the assay plate. The concentration of substrate used is based on being 2 to 5 fold-above the K_{M} for the enzyme.

Test compound stocks, 50 or 10 mM in DMSO, are diluted 1:50 or 1:10 respectively, in DMSO for a 1 mM working solution (or diluted as required with assay buffer depending on the compound to be tested to create a working solution).

The assay is performed at room temperature by adding these components in the following order:

1) Test compound: 100 μl of working solution

2) Substrate: 50 μl of working solution

3) Enzyme: 50 μl of working solution

An internal reference compound (i.e. a known cathepsin S inhibitor) is used on each plate.

After all the reagents are added to the assay plate, the plates (black 96 well, Costar™, Corning Incorporated, Corning, N.Y.) are read in a fluorescent plate reader (Flexstation, Molecular Devices Corporation, Sunnyvale Calif.), at λₑₓ 340 and λₑₘ 400 according to conventional methods. Data are collected every 5 minutes for up to 60 minutes. Only one set of data is used to calculate the percent inhibition or IC₅₀ of the tested compounds. This is chosen based upon the data set containing an internal reference standard being closest to the expected IC₅₀. This is typically between 15 to 20 minutes. If the internal reference is above or below 2 SD of the average value, the data generated are not used to calculate the compounds inhibitory capacity.

This plate methodology is ideally suited to high throughput screening. It should be noted that this assay is sensitive to DMSO. Thus, it is critical that in the preparation of substrate and test compound that less than 3% DMSO is used in the final assay mixture.

### EXAMPLE 5

### Antisense Oligonucleotides to Cathepsin S

Antisense oligonucleotides (ASOs) useful to inhibit gene expression, including the expression of cathepsin S, may be made according to conventional methods. In addition, one may employ additional methodologies, for example:

Synthesis of ASOs:

ASOs against cathepsin S may be fully phosphorothioated or fully phosphodiesterl8-mers with nucleotides at both ends modified with MOE (methoxy ethoxy) groups. These may be synthesized using phosphoramidite chemistry, HPLC-purified and characterized by electrospray mass spectrometry and capillary gel electrophoresis according to conventional methods. ASOs, each with a GC content between 38 and 72%, may be selected and synthesized complementary to parts of the coding region of, for example, rat or human cathepsin S. For mismatch-containing control oligonucleotides, the approximate base composition of the match oligonucleotides may be maintained. Additionally, two control ASOs may be selected, e.g., one for rat GAPDH coding regions and a second random synthetic ASO. The format of the anti-rat-GAPDH oligonucleotide may be the same as for anti-cathepsin S oligonucleotides; the synthetic oligonucleotide may have its MOE ribonucleotide modifications at both ends of the sequence with phosphorothioate or phosphodiester DNA residues in the middle.

Transfection Protocol:

Twenty four hours before transfection, 2×10⁵ cells e.g., Chinese Hamster Ovary cells (ICN Pharmaceuticals Ltd., Basingstoke, Hampshire, U.K.) in a volume of 2 ml per well (F12 Nutrient mix (DMEM), 100 unit/millilitre Penicillin, 100 micrograms per millilitre streptomycin, 2 millimolar L-Glutamine, 10% fetal bovine serum (GIBCO-BRL, Rockville, Md.)) may be plated into 6-well plates and cultured in 5% CO₂ to yield 70-80% confluency. On the day of transfection, a 2 fold stock transfection solution is prepared by diluting Lipofectin™ into serum-free OptiMEM (GIBCO-BRL, Rockville, Md.) (3 microliters Lipofectin™ per 100 nM desired final oligonucleotide concentration into 1 ml OptiMEM) and incubating for 15 minutes at room temperature. This solution is then combined 1:1 with a 2 fold ASO-solution containing twice the desired final amount of ASO in OptiMEM. After incubating the transfection mixture for 15 minutes at room temperature to form the transfection complex, 2 ml is added to each of the previously aspirated well of cells. A Lipofectin™ reagent-only control and a normal cell control (untreated) may also be included. After incubation for 4 hours at 37° C., 500 microliters of 50% FBS in MEM (GIBCO-BRL) is then added to each well to obtain a final FBS concentration of 10%. The cultures are then incubated at 37° C. in a humidified incubator with 5% CO₂ for 24 hours for mRNA harvest or 48 hours for protein harvest and electrophysiology.

Real-Time Quantitative PCR mRNA Analysis:

Total RNA may be isolated with the RNeasy 96 Kit (Qiagen, GmBH, Germany) according to the manufacturer's protocol. The RNA samples are individually diluted to 1 ng/L. Five nanograms of RNA for each sample are then mixed with gene-specific detection primers (easily determined by one of skill in the art) and with the appropriate reagents from the real-time quantitative PCR reaction kit PLATINUM® Quantitative RT-PCR THERMOSCRIPT™ One-Step System (Gibco-BRL, Rockville, Md.) and run according to manufacturer's protocol. The rat cathepsin S primers with the appropriate sequences may be purchased from PE Biosystems. GAPDH may be chosen as a control gene for comparisons. The same RNA samples may be run with rat GAPDH primers from the TaqMan® Rodent GAPDH Control Reagents Kit (PE Biosystems). The sequence-specific fluorescent emission signal can be detected using the ABI PRISM™ 7700 Sequence Detector (PE Applied Biosystems, Foster City, Calif.). Along with the samples, a standard from dilutions of pure template mRNA is run to obtain absolute concentrations per inserted amount of total RNA.

Testing the Cathepsin S Antisense in Animal Models of Neuropathic Pain:

Rats (e.g. Wistar) may be intrathecally cannulated in the lumbar or thoracic region of the spinal cord with a catheter attached to a minipump delivery system according to conventional methods. Antisense, missense oligos or vehicle may then be delivered for up to 7 days at a desired concentration to allow cell bodies within the spinal cord and the dorsal root ganglia to take up the oligos or vehicle. Nerve injury may be performed either before or after cannulation according to the pain models described herein. Mechanical hyperalgesia, allodynia etc may be measured in the usual way to assess the effect of cathepsin S antisense oligonucleotides in reversal of hyperalgesia.

What is claimed is:

1. A method to treat or ameliorate chronic pain comprising administering to a subject in need thereof an effective amount of a cathepsin S modulator.

2. The method of claim 1 wherein said chronic pain is chronic neuropathic pain.

3. The method of claim 1 wherein said cathepsin S modulator inhibits the enzyme activity of cathepsin S in said subject.

4. The method of claim 1 wherein said cathepsin S modulator inhibits cathepsin S gene expression in said subject.

5. The method of claim 1 wherein said cathepsin S modulator is a compound belonging to a of class of compound referred to as N-heteroaryl-carbonitrile cathepsin inhibitors.

6. The method of claim 1 wherein said modulator is [7-(2,2-Dimethyl-propyl)-6-thiophen-2-ylmethyl-7.H.-pyrrolo[2,3-.d.]pyrimidine-2-carbonitrile] in free or pharmaceutically acceptable salt forms.

7. The method of claim 1 wherein said modulator is 3-[(4-morpholinylcarbonyl)-phenylalanylamido]-1-fluoro-5-phenyl-2-pentanone.

8. The method of claim 1 wherein said modulator comprises any one or more substances selected from the group consisting of antisense oligonucleotides, triple helix DNA, ribozymes, RNA aptamers and double stranded RNA wherein said substances are designed to inhibit cathepsin S gene expression.

9. The method of claim 1 wherein said modulator comprises one or more antibodies to cathepsin S, or fragments thereof, wherein said antibodies or fragments thereof can inhibit cathepsin S enzyme activity.

10. A method to treat or ameliorate chronic pain comprising administering to a subject in need thereof a pharmaceutical composition comprising an effective amount of a cathepsin S modulator.

11. The method of claim 10 wherein said chronic pain is chronic neuropathic pain.

12. The method of claim 10 wherein said cathepsin S modulator inhibits the enzyme activity of cathepsin S in said subject.

13. The method of claim 10 wherein said cathepsin S modulator inhibits cathepsin S gene expression in said subject.

14. The method of claim 10 wherein said cathepsin S modulator is a compound belonging to a of class of compound referred to as N-heteroaryl-carbonitrile cathepsin inhibitors.

15. The method of claim 10 wherein said modulator is [7-(2,2-Dimethyl-propyl)-6-thiophen-2-ylmethyl-7.H.-pyrrolo[2,3-.d.]pyrimidine-2-carbonitrile] in free or pharmaceutically acceptable salt forms.

16. The method of claim 10 wherein said modulator is 3-[(4-morpholinylcarbonyl)-phenylalanylamido]-1-fluoro-5-phenyl-2-pentanone.

17. The method of claim 10 wherein said modulator comprises any one or more substances selected from the group consisting of antisense oligonucleotides, triple helix DNA, ribozymes, RNA aptamers and double stranded RNA wherein said substances are designed to inhibit cathepsin S gene expression.

18. The method of claim 10 wherein said modulator comprises one or more antibodies to cathepsin S, or fragments thereof, wherein said antibodies or fragments thereof can inhibit cathepsin S enzyme activity.

19. A method to identify modulators useful to treat or ameliorate chronic pain comprising assaying for the ability of a candidate modulator to inhibit cathepsin S activity.

20. The method of claim 19 wherein said method further comprises assaying for the ability of an identified cathepsin S inhibitory modulator to reverse the pathological effects observed in animal models of chronic pain and/or in clinical studies with subjects with chronic pain.

21. The method according to claim 19 wherein said chronic pain is chronic neuropathic pain.

22. A method to identify modulators useful to treat or ameliorate chronic pain comprising assaying for the ability of a candidate modulator to inhibit cathepsin S gene expression.

23. The method according to claim 22 wherein said method further comprises assaying for the ability of an identified inhibitory modulator to reverse the pathological effects observed in animal models of chronic pain and/or in clinical studies with subjects with chronic pain.

24. The method according to claim 22 wherein said chronic pain is chronic neuropathic pain.

25. A pharmaceutical composition comprising a cathepsin S modulator in an amount effective to treat or ameliorate chronic pain in a subject in need thereof.

26. The pharmaceutical composition according to claim 25 wherein said chronic pain is chronic neuropathic pain.

27. The pharmaceutical composition according to claim 25 wherein said modulator inhibits the enzyme activity of cathepsin S.

28. The pharmaceutical composition according to claim 25 wherein said modulator inhibits cathepsin S gene expression.

29. The pharmaceutical composition according to claim 25 wherein said modulator is a compound belonging to a class of compounds referred to as N-heteroaryl-carbonitrile cathepsin inhibitors.

30. The pharmaceutical composition of claim 25 wherein said modulator is [7-(2,2-Dimethyl-propyl)-6-thiophen-2-ylmethyl-7.H.-pyrrolo[2,3-.d.]pyrimidine-2-carbonitrile] in free or pharmaceutically acceptable salt forms.

31. The pharmaceutical composition of claim 25 wherein said modulator is 3-[(4-morpholinylcarbonyl)-phenylalanylamido]-1-fluoro-5-phenyl-2-pentanone.

32. The pharmaceutical composition of claim 25 wherein said modulator comprises any one or more substances selected from the group consisting of antisense oligonucleotides, triple helix DNA, ribozymes, RNA aptamer and double stranded RNA wherein said substances are designed to inhibit cathepsin S gene expression.

33. The pharmaceutical composition of claim 25 wherein said modulator comprises one or more antibodies to cathepsin S, or fragments thereof, wherein said antibodies or fragments thereof can inhibit cathepsin S enzyme activity.

34. A method to diagnose subjects suffering from chronic pain who may be suitable candidates for treatment with cathepsin S modulators comprising assaying mRNA levels of this protein in a biological sample from said subject wherein subjects with increased levels compared to controls would be suitable candidates for cathepsin S modulator treatment.

35. A method to diagnose subjects suffering from chronic pain who may be suitable candidates for treatment with cathepsin S modulators comprising detecting levels of this protein in a biological sample from said subject wherein subjects with increased levels compared to controls would be suitable candidates for cathepsin S modulator treatment.

36. A method to treat or ameliorate chronic pain comprising:
(a) assaying for cathepsin S mRNA and/or protein levels in a subject; and,
(b) administering to a subject with increased levels of cathepsin S mRNA and/or protein levels compared to controls a cathepsin S modulator in an amount sufficient to treat or ameliorate the pathological effects of chronic pain.

37. The method of claim 36 wherein said chronic pain is chronic neuropathic pain.

38. A diagnostic kit for detecting mRNA levels and/or protein levels of cathepsin S in a biological sample, said kit comprising:
(a) a polynucleotide of cathepsin S or a fragment thereof;
(b) a nucleotide sequence complementary to that of (a);
(c) a cathepsin S polypeptide, or a fragment thereof; or
(d) an antibody to a cathepsin S polypeptide
wherein components (a), (b), (c) or (d) may comprise a substantial component.


SEQUENCE LISTING (5 sequences)

SEQ ID NO: 1 - DNA, 20 bp, Artificial Sequence
  primer
gcaatggtggcttcatgaca

SEQ ID NO: 2 - DNA, 23 bp, Artificial Sequence
  primer
acatttctgatccatggctttgt

SEQ ID NO: 3 - DNA, 22 bp, Artificial Sequence
  primer
tgggaatgcactcatacgatct

SEQ ID NO: 4 - DNA, 20 bp, Artificial Sequence
  primer
ccactggctgggaactctca

SEQ ID NO: 5 - DNA, 75 bp, Artificial Sequence
  primer
aaacgacggcacttcgaaattaatacgactcactatagggagacctttttttttttttttttttttttttttttt